(19) United States
(12) Patent Application Publication
(10) Pub. No.: US 2026/0273184 A1
(43) Pub. Date: Sep. 17, 2026

(54) TECHNIQUES FOR DETERMINING AUTOMATED INSULIN DELIVERY DOSAGES

(71) Applicant: INSULET CORPORATION, Acton, MA (US)

(72) Inventors: Joon Bok LEE, Acton, MA (US); Yibin ZHENG, Hartland, WI (US); Steven CARDINALI, Tewksbury, MA (US); Jason O'CONNOR, Acton, MA (US)

(21) Appl. No.: 19/560,926

(22) Filed: Mar. 9, 2026

Related U.S. Application Data
(63) Continuation of application No. 17/027,191, filed on Sep. 21, 2020, now Pat. No. 12,569,619.

(51) Int. Cl. A61M 5/172; A61B 5/00; A61B 5/145; A61K 38/28; A61M 5/142; A61M 5/168

(52) U.S. Cl. CPC A61M 5/1723; A61M 5/16804; A61B 5/14532; A61B 5/7275; A61K 38/28; A61M 2005/14208; A61M 2205/3327; A61M 2205/50; A61M 2205/52; A61M 2230/201

(57) ABSTRACT

Methods and apparatuses for performing an insulin infusion process are described. For example, an apparatus may include at least one memory and logic coupled to the at least one memory. The logic may operate to determine a basal parameter for a patient based on a type 2 diabetes (T2D) multiple daily injection (MDI) information of the patient, the basal parameter indicating a basal infusion rate, determine an additional insulin (I_{add}) value based on a mean blood glucose difference (BG_{diff}) information associated with the patient, determine an insulin volume to infuse into the patient based on the basal parameter and I_{add}, and administer the insulin volume to the patient. Other embodiments are described.

### RELATED APPLICATIONS

This application is a continuation of U.S. patent application Ser. No. 17/027,191, filed Sep. 21, 2020, the contents of which are incorporated herein by reference in their entirety.

### FIELD OF THE INVENTION

The present disclosure generally relates to automated insulin delivery processes, and, more particularly, to processes for automated insulin delivery to treat diabetes.

### BACKGROUND

Diabetes mellitus is a serious medical condition caused by an inability to adequately control blood glucose levels. Typical treatments involve injecting affected individuals with the hormone insulin in an attempt to maintain blood glucose values within a desired, healthy range. Type 1 diabetes mellitus (T1D) results from an autoimmune response in which the immune system attacks pancreatic beta cells so that they no longer produce insulin. For type 2 diabetes mellitus (T2D), the pancreas may produce insulin, but it is either not a sufficient amount and/or the body's cells do not adequately respond to the insulin.

Treatment advances for TID patients have provided for automatic insulin delivery (AID) systems to control patient insulin levels. For example, an AID system may include a wearable insulin pump that operates to automatically inject insulin into the patient periodically or based on an event (for instance, user input, a determination that the patient blood sugar is below a threshold value, and/or the like). The dosage of insulin injected via the AID system may be determined based on historical information, blood glucose information measured using AID system sensors, and/or other factors (for instance, weight, ketones, manual information (for example, the patient is having a meal or the patient is exercising)), and/or the like.

Patients with advanced T2D also require regular insulin infusion. However, conventional treatments for T2D patients generally involve manual blood glucose measurements (for instance, finger-stick measurements) and needle injections performed by the patient. Conventional AID systems, which have been designed to treat TID patients, are not able to directly operate for T2D patients because glucose metabolism and insulin kinetics are significantly different for T2D patients compared with TID patients. Accordingly, it would be beneficial and advantageous to have a system, a device and/or a technique for safely and effectively providing automated delivery methods for providing insulin to T2D patients.

### BRIEF DESCRIPTION OF THE DRAWINGS

FIG. 1 illustrates a first exemplary operating environment in accordance with the present disclosure;

FIG. 2 illustrates a second exemplary operating environment in accordance with the present disclosure;

FIG. 3 illustrates a first logic flow in accordance with the present disclosure;

FIG. 4 illustrates a second logic flow in accordance with the present disclosure

FIG. 5 illustrates a third logic flow in accordance with the present disclosure

FIG. 6 illustrates a fourth logic flow in accordance with the present disclosure; and

The drawings are not necessarily to scale. The drawings are merely representations, not intended to portray specific parameters of the disclosure. The drawings are intended to depict example embodiments of the disclosure, and therefore should not be considered as limiting in scope. In the drawings, like numbering represents like elements.

### DETAILED DESCRIPTION

The described technology generally relates to an insulin infusion process for automatically infusing a patient with insulin. In some embodiments, an insulin infusion process may be used with additional processes, algorithms, or computer applications that manage blood glucose levels and/or other forms of insulin therapy. Such processes or algorithms may generally be referred to as an “artificial pancreas” (AP) system or application or an automatic insulin delivery (AID) system or application that may operate to provide automatic delivery of insulin. In some embodiments, the automatic delivery of insulin may be based, at least in part, on blood glucose information. In some embodiments, the blood glucose information may be obtained via sensor input, such as data measured via a continuous glucose monitor (CGM) device or sensor and/or from manual measurement (for instance, via a finger-stick measurement manually entered by a user).

Conventional AID systems have generally been developed to deliver insulin based on the needs of patients with Type 1 diabetes (TID). However, patients with advanced Type 2 diabetes (T2D) also require regular insulin infusion and, accordingly, would benefit from an AID system that is tailored specifically to the needs of T2D patients that is efficient, accurate, and does not require complex intervention, such as manual estimations of blood glucose levels and/or insulin needs. There is significant difficulty in designing an optimal AID system for T2D patients using existing technology because, among other reasons, glucose metabolism, insulin kinetics, and other disease characteristics of T2D patients are significantly different than those for TID patients. Therefore, AID systems developed to manage TID cannot be directly transferred to treating T2D patients.

Accordingly, some embodiments may provide processes, devices, techniques, methods, and/or other technology for operating an AID system for implementing insulin therapy to treat T2D patients. For example, some embodiments may provide an insulin delivery process configured to utilize multiple daily injection (MDI) recommendations for T2D patients operative to provide a safe, accurate, and effective AID system that can reduce the burden of insulin delivery for T2D patients. Although T2D treatments and T2D patients may be used in examples in the present disclosure, embodiments are not so limited, as the insulin delivery processes of the present disclosure may be used for TID and/or other conditions according to some embodiments.

A T2D patient typically receives basal insulin delivery via MDI (however, without compensation from bolus insulin for excursions, such as meals or food ingestions). Accordingly, some embodiments may provide an insulin delivery process operative to convert MDI therapy information for T2D patients into continuous subcutaneous insulin infusion (CSII) information, such as CSII rates for use with an AID system. In various embodiments, an insulin delivery process may be operative to set maximum additional insulin delivery based, for example, on differences between mean blood glucose and target blood glucose information. In exemplary embodiments, an insulin delivery process may be operative to determine and adjust safety constraints for providing insulin to T2D patients via an AID system. For example, insulin delivery processes may be configured to determine, adjust, or tune safety constraints to avoid hypoglycemia specifically for T2D patients, for example, accounting for the different characteristics and lower risks of hypoglycemia for T2D patients compared with TID patients. In various embodiments, an insulin delivery process may be operative to deliver insulin using an AID system based on finger-stick blood glucose measurements (for example, instead of or in addition to CGM measurements).

Therefore, insulin infusion processes according to some embodiments may provide multiple technological advantages and technical features over conventional systems, including improvements to computing technology. One non-limiting example of a technological advantage may include determining AID processes based, at least in part, on conventional, manual T2D treatment information (for instance, MDI and/or total daily insulin (TDI) information). In this manner, T2D patients may be able to utilize automated processes and devices for treating T2D, experiencing the same or similar advantages for treatment, user experience, convenience, accuracy, efficiency, and/or the like available for AID treatment regimens. In another non-limiting example of a technological advantage, the computing technology of an AID device (including, for example, the wearable infusion device and/or a controller computing device) may be improved by being able to be controlled to implement an insulin infusion process according to some embodiments for treating T2D using the same or similar hardware (for example, conventionally used for TID treatments). In a further non-limiting technological advantage, an AID system may operate to infuse a T2D patient with insulin using safety protocols and constraints specific for T2D patients without negatively affecting performance.

In addition, some embodiments may provide one or more practical applications of insulin infusion processes, algorithms, and/or the like described in the present disclosure. Illustrative and non-limiting practical applications may include treating diabetes, such as T2D, infusing a safe amount of insulin into a diabetic patient, operating an infusion device (for instance, an AID device) according to an insulin infusion process to provide a prescribed or pre-determined amount of insulin that is safe and effective for a diabetic patient, converting conventional manual T2D information (for instance, MDI) into information configured to be used with an automated system, such as an AID system, and/or the like. Other technological advantages, improvements, and/or practical applications are provided by embodiments described in the present disclosure and would be understood by persons of skill in the art. Embodiments are not limited in this context.

In this description, numerous specific details, such as component and system configurations, may be set forth in order to provide a more thorough understanding of the described embodiments. It will be appreciated, however, by one skilled in the art, that the described embodiments may be practiced without such specific details. Additionally, some well-known structures, elements, and other features have not been shown in detail, to avoid unnecessarily obscuring the described embodiments.

In this Detailed Description, references to “one embodiment,” “an embodiment,” “example embodiment,” “various embodiments,” etc., indicate that the embodiment(s) of the technology so described may include particular features, structures, or characteristics, but more than one embodiment may and not every embodiment necessarily does include the particular features, structures, or characteristics. Further, some embodiments may have some, all, or none of the features described for other embodiments.

As used in this description and the claims and unless otherwise specified, the use of the ordinal adjectives “first,” “second,” “third,” etc. to describe an element merely indicate that a particular instance of an element or different instances of like elements are being referred to, and is not intended to imply that the elements so described must be in a particular sequence, either temporally, spatially, in ranking, or in any other manner.

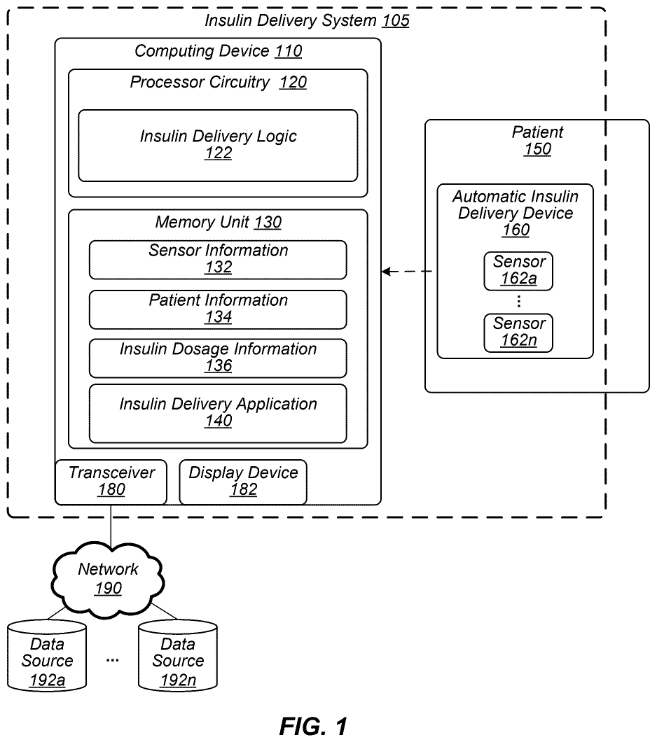

FIG. 1 illustrates an example of an operating environment 100 that may be representative of some embodiments. As shown in FIG. 1, operating environment 100 may include an insulin infusion system 105. In various embodiments, insulin infusion system 105 may include a computing device 110 that, in some embodiments, may be communicatively coupled to network 190 via a transceiver 180. Computing device 110 may be or may include a display device 182 and one or more logic devices, including, without limitation, a server computer, a client computing device, a personal computer (PC), a workstation, a laptop, a notebook computer, a smart phone, a tablet computing device, a personal diabetes management (PDM) device, and/or the like. Embodiments are not limited in this context.

Insulin infusion system 105 may include or may be communicatively coupled to an automatic insulin delivery (AID) device 160 configured to deliver insulin (and/or other medication) to patient 150. AID device 160 may be a wearable device. For example, AID device 160 may be directly coupled to patient 150 (for instance, directly attached to a body part and/or skin of the user via an adhesive and/or other attachment component).

AID device 160 may include a number of components to facilitate automated delivery of insulin to patient 150. For example, AID device 160 may include a reservoir for storing insulin, a needle or cannula for delivering insulin into the body of the person, and a pump for transferring insulin from the reservoir, through the needle or cannula, and into the body of the patient. AID device 160 may also include a power source, such as a battery, for supplying power to the pump and/or other components of automatic insulin delivery device 160. Embodiments are not limited in this context, for example, as AID device 160 may include more or less components.

AID device 160 may store and provide any medication or drug to the user. In various embodiments, AID device 160 may be or may include a wearable AID device. For example, AID device 160 may be the same or similar to an OmniPod® device or system provided by Insulet Corporation of Acton, Massachusetts, United States, for example, as described in U.S. Pat. Nos. 7,303,549; 7,137,964; and/or 6,740,059, each of which is incorporated herein by reference in its entirety.

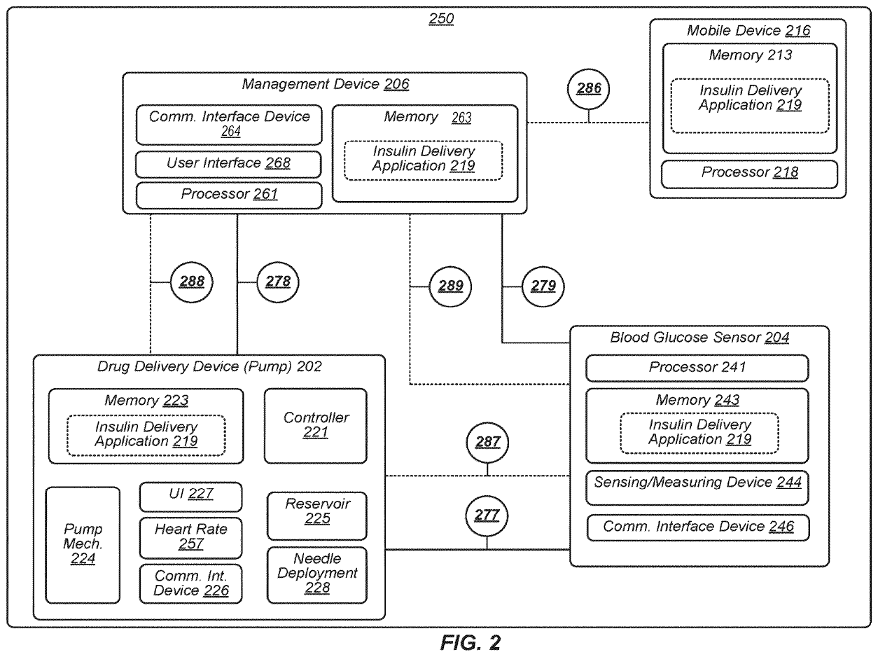

In some embodiments, computing device 110 may be a smart phone, PDM, or other mobile computing form factor in wired or wireless communication with automatic insulin delivery device 160. For example, computing device 110 and AID device 160 may communicate via various wireless protocols, including, without limitation, Wi-Fi (i.e., IEEE 802.11), radio frequency (RF), Bluetooth™, Zigbee™, near field communication (NFC), Medical Implantable Communications Service (MICS), and/or the like. In another example, computing device 110 and adjustment compliance device may communicate via various wired protocols, including, without limitation, universal serial bus (USB), Lightning, serial, and/or the like. Although computing device 110 (and components thereof) and AID device 160 are depicted as separate devices, embodiments are not so limited. For example, in some embodiments, computing device 110 and AID device 160 may be a single device. In another example, some or all of the components of computing device 110 may be included in automatic insulin delivery device 160. For example, AID device 160 may include processor circuitry 120, memory unit 130, and/or the like. In some embodiments, each of computing device 110 and AID device 160 may include a separate processor circuitry 120, memory unit 130, and/or the like capable of facilitating insulin infusion processes according to some embodiments, either individually or in operative combination. Embodiments are not limited in this context (see, for example, FIG. 2).

AID device 160 may include or may be communicatively coupled to one or more sensors 162a-n operative to detect, measure, or otherwise determine various physiological characteristics of patient 150. For example, a sensor 162a-n may be or may include a CGM sensor operative to determine blood glucose measurement values of patient 150. In another example, a sensor 162a-n may include a heart rate sensor, temperature sensor, and/or the like.

Computing device 110 (and/or automatic insulin delivery device 160) may include a processor circuitry 120 that may include and/or may access various logics for performing processes according to some embodiments. For instance, processor circuitry 120 may include and/or may access an insulin delivery logic 122. Processing circuitry 120, insulin delivery logic 122, and/or portions thereof may be implemented in hardware, software, or a combination thereof. The functions, processes, algorithms, and/or the like (for example, an insulin infusion process) described according to some embodiments may be performed by processor circuitry and/or insulin delivery logic 122 (for example, via executing insulin delivery application 140) by computing device 110, automatic insulin delivery device 160, and/or a combination thereof. The processing circuitry 120, memory unit 130, and associated components are depicted within computing device 110 to simplify FIG. 1 (for instance, all or a portion of processing circuitry 120, memory unit 130, and associated components may be arranged within automatic insulin delivery device 160). Accordingly, embodiments of functionality, processes (for instance, an insulin infusion process), and/or the like described in the present disclosure with respect to computing device 110 and/or components thereof may be performed in whole or in part by automatic insulin delivery device 160.

As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, FG circuitry, variations thereof, combinations of any of the foregoing, and/or the like.

Although insulin delivery logic 122 is depicted in FIG. 1 as being within processor circuitry 120, embodiments are not so limited. For example, insulin delivery logic 122 and/or any component thereof may be located within an accelerator, a processor core, an interface, an individual processor die, implemented entirely as a software application (for instance, an insulin delivery application 140) and/or the like.

Memory unit 130 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 130 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

Memory unit 130 may store various types of information and/or applications for an insulin infusion process according to some embodiments. For example, memory unit 130 may store sensor information 132, patient information 134, insulin dosage information 136, and/or insulin delivery application 140. In some embodiments, sensor information 132, patient information 134, insulin dosage information 136, insulin delivery application 140, and/or portions thereof may be stored in one or more data stores 192a-n accessible to computing device 110 (and/or automatic insulin delivery device 160) via network 190.

In some embodiments, insulin delivery application 140 may be or may include an application being executed on computing device 110 and/or AID device 160 (including a mobile application, “mobile app,” or “app” executing on a mobile device form factor). For example, in various embodiments, insulin delivery application 140 may be or may include an application the same or similar to the Omnipod® Mobile App, Glooko, Omnipod® DASH™ PDM software, and/or the like provided by Insulet Corporation of Acton, Massachusetts, United States. In addition or in the alternative, insulin delivery application 140 may be or may include an application operative to control components of automatic insulin delivery device (for instance, a pump, sensors 162a-n, and/or the like) to infuse patient 150 with insulin, such as an AID application. For example, insulin delivery application 140 may be or may include an AID application to monitor patient blood glucose values, determine an appropriate level of insulin based on the monitored glucose values (e.g., blood glucose concentrations and/or blood glucose measurement values) and other information, such as user-provided information, including, for example, carbohydrate intake, exercise times, meal times, and/or the like, and perform an insulin infusion process according to some embodiments to maintain a user's blood glucose value within an appropriate range.

In some embodiments, sensor information 132 may include information determined via sensors 162a-n. For example, sensor information 132 may include CGM information (for instance, blood glucose concentrations or other blood glucose measurement values), temperature information, heart rate information, and/or the like. In exemplary embodiments, sensor information 132 may include historical information, for instance, historical blood glucose values of patient 150. In various embodiments, patient information 134 may include information associated with patient 150. Non-limiting examples of patient information 134 may include demographic information, physical information (for instance, height, weight, and/or the like), diabetes condition information (for instance, type of diagnosed diabetes (TID or T2D)), insulin needs (for instance, MDI information, TDI information, insulin types, and/or the like), activity information (for instance, meals and/or meal times, carbohydrate intake, exercise information, and/or the like), insulin sensitivity information, and/or the like. In some embodiments, at least a portion of patient information 134 may be manually entered by patient 150 or a caregiver, for example, via a user interface of insulin delivery application 140. In some embodiments, patient information 134 may include historical information, such as historical values associated with mealtimes, carbohydrate intake, exercise times, and/or the like.

In some embodiments, insulin dosage information 136 may include information used to perform an insulin infusion process via AID device 160 according to some embodiments. Non-limiting examples of insulin infusion information may include MDI information, TDI information, CGM information, basal dosage information, basal rate information, basal parameter(s), current glycated hemoglobin (HbA1c or “A1C”) information, target A1C information, blood glucose difference (for instance, BG_{diff}), correction factor parameter(s) (CF), maximum additional insulin delivery information (I_{add}), AID process or algorithm information, safety constraint information, adjustment factor(s) (Fₛ), thresholds (for example, total insulin delivery during a certain duration, insulin-on-board (IOB), IOB decay rate (D(t)), and/or the like), change in basal (for example, Δb(i)), insulin sensitivity factors, constants, tunable parameters, and/or the like.

Insulin delivery logic 122, for example, implemented via insulin delivery application 140 being executed by processor circuitry 120, may operate to perform an insulin infusion process according to some embodiments to infuse a patient with insulin. In various embodiments, the insulin infusion process may be configured for a T2D patient.

In some embodiments, the insulin infusion process may operate to determine insulin dosage information 136 in the form of a basal parameter for patient 150. The basal parameter may be used by the insulin infusion process, for example, via an associated AID algorithm, to deliver a dosage of insulin to patient 150. For example, the basal parameter may be or may be used for determining continuous insulin infusion (CII) rate. A non-limiting example of a CII may be or may include continuous subcutaneous insulin infusion (CSII) information. However, embodiments are not limited to subcutaneous infusion (for instance, insulin delivery may occur at the dermal layer of the skin of patient 150). In some embodiments, the basal parameter may be the basal insulin needs of patient 150.

In some embodiments, insulin delivery logic 122 may implement the insulin infusion process to convert MDI insulin quantities of patient 150 into CII (or CSII) for use in an AID algorithm. For example, T2D patients may utilize one or more daily injections (MDI) of insulin per day of a fixed quantity of (long-acting) insulin for each injection. For instance, patient 150 may be a T2D patient having an MDI protocol requiring two injections of 20 units (U) of long-acting insulin, for a TDI total of 40 U per day. In various embodiments, the total amount of insulin (TDI or total daily dosage (TDD)) to be delivered may be converted into a basal parameter. For example, in some embodiments, it may be assumed that T2D patients often do not bolus for their meals such that the entirety of the insulin deliveries each day can be considered to be their basal needs, instead of the typical assumption of utilizing 50% of the total insulin deliveries as their basal needs (for instance, for TID patients used in conventional AID algorithms).

The following Equation (1) may be used to determine TDI based on MDI:

$$TDI = {\sum }_{n = 1}^{{n}_{max}} {MDI}_{n} ,$$

where MDIₙ is the n^{th} injection of patient 150. The following Equation (2) may be used to determine the basal parameter based on TDI:

$$basal = \frac{TDI}{24} .$$

In various embodiments, the basal parameter may be used as input to an AID algorithm, for example, to determine an infusion rate, total infusion dosage, and/or the like. In Equation (2), the value 24 may indicate a 24-hour period for the basal parameter. Other values may be substituted for other time periods (for instance, 48 for ½ hour time periods, 144 for 10-minute time periods, 288 for 5-minute time periods, and so on). For example, for a patient having a TDI of 45 units, the basal parameter may be about 1.875 units/hour for an AID algorithm infusion rate.

In some embodiments, the insulin infusion process may determine a maximum daily dosage of insulin. In various embodiments, the maximum daily dosage may be determined based, at least in part, on target health conditions for patient 150. For example, in one embodiment, the maximum daily dosage may be determined based on a target A1C for patient 150. A1C is an important factor in assessing the quality of blood glucose control for people with T2D. In general, A1C is a percentage indicating patient blood sugar levels over a previous time period, typically two to three months. A normal A1C (for instance, of a non-diabetic individual) may be a value less than about 6.0%. A target A1C for T2D patients may be about 7.0%; however, each patient may have their own target A1C. Diabetes management of people with T2D often have a target A1C, for example, that is lower than their current A1C.

In some embodiments, the difference between the current A1C and the target A1C may be converted into a difference in mean blood glucose (BG_{diff}) according to the following Equation (3):

$$A 1 {C}_{c u r r e n t} - A 1 {C}_{t a r g e t} = \frac{B {G}_{diff} + 68.8}{31.5} .$$

In various embodiments, the basal needs of patient 150 (for instance, the basal parameter) may be used to estimate a correction factor (CF) parameter (for instance, for T2D patients). In some embodiments, CF may be determined by converting the basal parameter to daily needs using an insulin sensitivity factor. In some embodiments, the insulin sensitivity factor may be the “1800 rule” or other similar sensitivity factor for indicating patient reactions to insulin (for instance, the 1800 rule may be used to determine how much a patient's blood sugar may drop for each unit of a particular type of insulin by dividing 1800 by the number of units of insulin delivered over an infusion time period). In various embodiments, each patient may have their own sensitivity factor. For example, in an embodiment, CF may be determined for the 1800 rule according to the following Equation (4):

$$C F = \frac{1800}{24 * basal} ,$$

where 24 indicates 24 hours per day (an infusion time period) and the 1800 (infusion sensitivity factor) may be substituted based on the particular insulin sensitivity used to determine CF.

In exemplary embodiments, the insulin infusion process may use the difference in mean BG (for instance, BG_{diff}) to determine an expected total additional insulin need per day (I_{add}) according to the following Equation (5):

$${I}_{add} = \frac{B {G}_{diff}}{C F} * 6 ,$$

where the value 6 is a tunable cycle parameter configured to represent the number of 4-hour cycles of insulin peak times per 24-hour period. Accordingly, Equation (5) may use different cycle parameter values other than 6 depending on, for instance, the specific treatment regimen and/or physiological characteristics of patient 150. In some embodiments, I_{add} may be insulin to be delivered to a patient in addition to the volume indicated by the basal parameter (for instance, bolus insulin requirements) to achieve diabetic therapy goals for patient 150.

In various embodiments, I_{add} may be be set as the maximum additional delivery of insulin allowed for patient 150 (for instance, the maximum allowed beyond TDI or basal). In some embodiments, I_{add} may be set as the maximum additional delivery of insulin allowed for patient 150 within a basal parameter time period (for instance, 24-hour time period) by AID device 160. For example, in various embodiments, the actual insulin delivery by AID device 160 may be defined according to the following Equation (6):

$${I}_{A I D} ( t ) = \{ \begin{matrix}{AID}_{a l g} ( t ) & {\sum }_{i = 1}^{T} {AID}_{alg} ( i ) − basal ( i ) \leq basal ⋆ T \\ b ( t ) & {\sum }_{i = 1}^{T} {AID}_{alg} ( i ) − basal ( i ) > basal ⋆ T\end{matrix} ,$$

where t is the current control cycle where the actual insulin delivery decision may be made, Tis the time unit of consideration to apply this limit (for instance, 1 for hour 1, 2 for hour 2, and so on), I_{AID}(t) is the amount of insulin to be infused into patient 150 at time t, AID_{alg}(t) is the amount of insulin determined for infusion according to the AID algorithm associated with AID device 160 at time t, and basal(t) is the basal units to be delivered at time t (for example, if basal is 30 units per day and t is 24 (for instance, infusion every hour), then basal(t) is 30/24=1.25). With reference to Equation (6), delivery of insulin according AID_{alg}(t) may occur if the maximum delivery limit has not been reached, and basal (b(t)) may be delivered if the maximum delivery limit has been reached. For example, I_{AID} may be or may be considered AID_{alg} if the sum of differences between AID_{alg}-basal is less than the threshold, and basal if AID_{alg}-basal is greater than the threshold. In some embodiments, the maximum delivery limit may be determined based on (basal parameter)+I_{add}. Accordingly, in some embodiments, the insulin infusion process may use Equation (6) to limit the automated insulin delivery to basal in a binary manner by comparing against the additional insulin delivery (I_{add}).

In one exemplary embodiment, the threshold for maximum insulin delivery allowed beyond the user's basal per 24 hour period can be set to 6 hours (T=6). In this example, defining T=6 may mean that the system cannot deliver more than 6 times basal, above the user's basal, in total, over the last 6 hours. Thus, at any cycle t, the system may take the sum of insulin deliveries in the last 6 hours, corresponding to the first term in the summation portion of Equation 6. In this example, for instance, the total delivery may be 15 U of insulin and basal may be 2 U/hour. Then, the sum of the total insulin delivery minus the total basal in the last 6 hours is 3 U. This is less than 12U, which is the stated maximum insulin delivery allowed above basal in this example. As a result, the system would deliver the full insulin delivery request as defined by AID_{alg}(t) for the current cycle t. On the other hand, if the total delivery was 25U, the user received 13U above basal, which exceeds the stated threshold of 12 U. In this case, the system would deliver the basal value b(t) instead of AID_{alg}(t) in the current cycle t.

In various embodiments, the insulin infusion process may include determining one or more safety constraints or safety constraint adjustments. In some embodiments, the safety constraint adjustments may be based, at least in part, on additional insulin delivery (I_{add}) requirements. In exemplary embodiments, the safety constraints may be adjusted to allow a continuous limitation of AID device 160 insulin delivery that still allows for delivery of an amount of insulin beyond I_{add} as necessary.

Specifically, safety constraints within an AID algorithm may be associated with, at least in some form, to the patient TDI. For example, because I_{add} may represent the additional total insulin delivery, the safety constraints may be adjusted (for example, relaxed) by an adjustment factor. For instance, the safety constraints may be relaxed by a percentage adjustment (Fₛ) based on the proportion of I_{add} versus the user's TDI, according to the following Equation (7):

$${F}_{s} = 1 + max ( 0 , \frac{{I}_{a d d}}{TDI} ) .$$

In some embodiments, the insulin infusion process may include a duration maximum threshold (or integral delivery) safety constraint. For example, the duration maximum factor may provide that the total insulin delivery for patient 150 during a certain duration (for example, 3 hours) cannot exceed a tunable factor (for instance, 9) times the basal parameter. The adjustment factor can affect (for example, increase) the duration maximum threshold, for instance, by multiplying the duration maximum threshold by Fₛ as provided in the following Equation (8):

$$Duration Maximum ( or {th}_{integral , m o d} ) = 9 * \frac{TDI}{48} * {F}_{s} ,$$

where 9 is the tunable factor and the value 48 may be modified based on the duration of interest.

In various embodiments, the insulin infusion process may include an insulin-on-board (IOB) safety constraint. In some embodiments, the IOB safety constraint may include determining insulin delivery in deviations from basal as a baseline. The IOB baseline (for instance, th_{IOB}) may be affected (for example, increased) by the adjustment factor Fₛ according to the following Equation (9):

$$t {h}_{IOB} = {\sum }_{i = 1}^{t} D ( t ) * ( I ( t ) - basal * {F}_{s} ) ,$$

where D(t) is an IOB decay rate tunable parameter.

In some embodiments, the insulin infusion process may operate AID device 160 insulin delivery based on manual blood glucose measurement instead of or in addition to CGM measurement processes. For example, the insulin infusion process may be configured to operate AID device 160 to automatically calculate adjustments in insulin delivery whenever patient 150 measures a fingerstick glucose value. In some embodiments, for instance, insulin delivery adjustments may be determined according to the following Equation (10):

$$Δ b ( i ) = ( B G ( i ) - target ) * TDI * C ,$$

where Δb(i) represents the change in basal of patient 150 based on the i^{th} fingerstick glucose (BG) measurement, and C is a tunable constant. In various embodiments, the manual fingerstick-based blood glucose meter (BG meter) may be communicatively coupled to AID device 160, for instance, via Bluetooth or another wired or wireless communication protocol. Accordingly, in various embodiments, the Δb(i) adjustment may be automatically implemented if AID device 160 is connected to the BG meter.

In various embodiments, tunable constant C may be determined assuming, for example, the insulin sensitivity of patient 150 (for instance, using the 1800 rule), and estimating or otherwise determining that the insulin delivery may occur over a delivery duration (for instance, 8 hours or the maximum duration of insulin action of patient 150) according to the following Equation (11):

$$C = \frac{1}{1800 * 8} ,$$

where the value 1800 may be substituted for other insulin sensitivity rules and 8 may be substituted for other maximum durations of insulin action.

In some embodiments, the Δb(i) adjustment may also be made asymmetric and/or dependent on the current blood glucose of patient 150. In various embodiments, for example, the Δb(i) adjustment may be modified by the value of the current blood glucose measurement according to the following Equation (12):

$$Δ b ( i ) = \{ \begin{matrix}BG ( i ) - target ) * TDI * C & BG ( i ) > 70 \\ 0 & BG ( i ) \leq 70\end{matrix} .$$

In exemplary embodiments, the insulin infusion process may modify the assumed rate t which insulin may take action based on the current blood glucose measurement of patient 150, for example, by adjusting the maximum durations of insulin action value (for instance, the “8” value in Equation (11)) of the C constant according to the following Equation (13):

$$C = \{ \begin{matrix}\frac{1}{1800 ⋆ 8} & BG ( i ) > target \\ \frac{1}{1800 ⋆ 7} & 70 < BG ( i ) \leq target \\ \frac{1}{1800 ⋆ 6} & BG ( i ) \leq 70\end{matrix} .$$

Accordingly, in some embodiments, insulin delivery may be adjusted (for instance, decreased) more rapidly if the blood glucose of patient 150 is below the target.

In various embodiments, instead of using a flat or semi-flat adjustment to a safety constraint, the safety constraints may be individually modified, for example, to correspond or match the insulin dynamics of T1D patients. For example, the need for additional insulin, combined with less likelihood of T2D patients experiencing hypoglycemic risk, may allow for direct adjustment of the time constants of the constraints in addition to the TDI baselines. For instance, the “3 hour” constraint of the integral delivery constraint may instead by set to a “2 hour” constraint; similarly, the insulin decay curve D(t) can be set to decay X % (accelerated decay factor) faster corresponding to Fₛ. For example, the accelerated decay factor may be about 1%, about 5%, about 10%, about 20%, about 30%, about 40%, about 50%, about 60%, about 70%, about 80%, about 90%, about 95%, or any values or ranges between any two of these values (including endpoints). Embodiments are not limited in this context.

FIG. 2 illustrates a second exemplary operating environment in accordance with the present disclosure. More specifically, FIG. 2 illustrates an example of an operating environment 200 implementing a drug delivery system that utilizes one or more examples of the insulin infusion process according to some embodiments, for example, as described with reference to FIGS. 1 and 3-6. In some embodiments, drug delivery system 250 may be an implementation of operating environment 100 of FIG. 1 (or vice versa).

As shown in FIG. 2, drug delivery system 250 may include a drug delivery device 202, a management device 206, and a blood glucose sensor 204. In some embodiments, drug delivery device 202 may be a wearable or on-body drug delivery device that is worn on the body of a patient or user. Drug delivery device 202 may include a pump mechanism 224 that may, in some examples be referred to as a drug extraction mechanism or component, and a needle deployment component 228. In various examples, the pump mechanism 224 may include a pump or a plunger (not shown).

Needle deployment component 228 may, for example, include a needle (not shown), a cannula (not shown), and any other fluid path components for coupling the stored liquid drug in reservoir 225 to the user. The cannula may form a portion of the fluid path component coupling the user to reservoir 225. After needle deployment component 228 has been activated, a fluid path (not shown) to the user is provided, and pump mechanism 224 may expel the liquid drug (for instance, insulin) from reservoir 225 to deliver the liquid drug to the user via the fluid path. The fluid path may, for example, include tubing (not shown) coupling wearable drug delivery device 202 to the user (e.g., tubing coupling the cannula to reservoir 225).

Wearable drug delivery device 202 may further include a controller 221 (for instance, the same or similar to processing circuitry 120) and a communications interface device 226. Controller 221 may be implemented in hardware, software, or a combination thereof. The controller 221 may, for example, be a processor, a logic circuit or a microcontroller coupled to a memory 223. Controller 221 may maintain a date and time as well as other functions (e.g., calculations or the like) performed by processors. Controller 221 may be operable to execute an AP or AID application, for example, insulin delivery application 219 stored in memory 223 that enables controller 221 to direct operation of drug delivery device 202. In addition, controller 221 may be operable to receive data or information indicative of physiological characteristics of the user from mobile device 216, blood glucose sensor 204, management device 206, and/or the like.

In some embodiments, drug delivery device 202 may include or may be communicatively coupled to a blood glucose sensor 204. In some embodiments, blood glucose sensor 204 may be a CGM sensor. In various embodiments, blood glucose sensor 204 may be a fingerstick-based blood glucose sensor. Blood glucose sensor 204 may be physically separate from drug delivery device 202 or may be an integrated component thereof. In various embodiments, blood glucose sensor 204 may provide controller 221 with data indicative of measured or detected blood glucose (BG) levels of the user. In some embodiments, a user may manually enter blood glucose measurements, for instance, measured via a fingerstick method into management device 206, mobile device 216, drug delivery device 202, and/or management device 206 for use by drug delivery device 202.

Management device 206 (for instance, a PDM) may be maintained and operated by the user or a caregiver of the user. Management device 206 may control operation of drug delivery device 202 and/or may be used to review data or other information indicative of an operational status of drug delivery device 202 or a status of the user. Management device 206 may be used to direct operations of drug delivery device 202. For example, management device 206 may be a dedicated personal diabetes management (PDM) device, a smartphone, a tablet computing device, other consumer electronic device including, for example, a desktop, a laptop, a tablet, or the like. Management device 206 may include a processor 261 and memory devices 263. In some embodiments, memory devices 263 may store an insulin delivery application 219 that may be or may include an AP or AID application including programming code that may implement delivery of insulin based on input from blood glucose sensor 204 (for instance, via a CGM-based blood glucose sensor 204 and/or a fingerstick-based blood glucose sensor 204) and/or manual user input.

In some embodiments, management device 206 may operate in cooperation with a mobile device 216. In various embodiments, mobile device 216 may include a memory 213 and a processor 218 as well as additional components and elements as discussed with reference to computing device 110 of FIG. 1. Memory 213 may store programming code as well as mobile computer applications, such as insulin delivery application 219

In an example, wearable drug delivery device 202 may be attached to the body of a user, such as a patient or diabetic, and may deliver any therapeutic agent, including any drug or medicine, such as insulin or the like, to a user. Wearable drug delivery device 202 may, for example, be a wearable device worn by the user. For example, wearable drug delivery device 202 may be directly coupled to a user (e.g., directly attached to a body part and/or skin of the user via an adhesive or the like). In an example, a surface of the wearable drug delivery device 202 may include an adhesive to facilitate attachment to a user. Wearable drug delivery device 202 may be referred to as a pump, or an insulin pump, in reference to the operation of expelling a drug from reservoir 225 for delivery of the drug to the user.

In an example, wearable drug delivery device 202 may include a reservoir 225 for storing the drug (such as insulin), a needle or cannula (not shown) for delivering the drug into the body of the user (which may be done subcutaneously, intraperitoneally, or intravenously), and a pump mechanism 224, or other drive mechanism, for expelling the stored insulin from the reservoir 225, through a needle or cannula (not shown), and into the user. Reservoir 225 may be operable to store or hold a liquid or fluid, such as insulin or another therapeutic drug. Pump mechanism 224 may be fluidly coupled to reservoir 225, and communicatively coupled to controller 221. Wearable drug delivery device 202 may also include a power source (not shown), such as a battery, a piezoelectric device, or the like, for supplying electrical power to pump mechanism 224 and/or other components (such as controller 221, memory 223, and communication interface device 226) of wearable drug delivery device 202.

In an example, blood glucose sensor 204 may be a CGM device communicatively coupled to the processor 261 or controller 221 and may be operable to measure a blood glucose value at a predetermined time interval, such as approximately every 5 minutes, or the like. Blood glucose sensor 204 may provide a number of blood glucose measurement values to the insulin delivery application 219 operating on the respective devices. In another example, blood glucose sensor 204 may be a manual blood glucose sensor measuring blood glucose in blood from a fingerstick method.

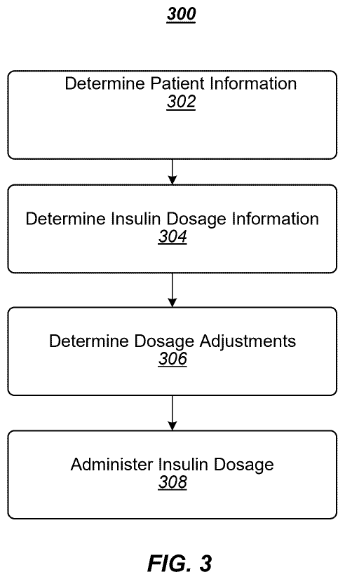

Wearable drug delivery device 202 may operate to provide insulin stored in reservoir 225 to the user based on information (for instance, insulin dosage information 136) determined via an insulin infusion process according to some embodiments. For example, wearable drug delivery device 202 may contain analog and/or digital circuitry that may be implemented as a controller 221 (or processor) for controlling the delivery of the drug or therapeutic agent. The circuitry used to implement controller 221 (the same or similar to processing circuitry 120) may include discrete, specialized logic and/or components, an application-specific integrated circuit, a microcontroller or processor that executes software instructions, firmware, programming instructions or programming code (for example, insulin delivery application 140 as well as the process examples of FIGS. 3-6) stored in memory 223, or any combination thereof. For example, controller 221 may execute a control algorithm, such an AID algorithm of insulin delivery application 219, that may make the controller 221 operable to cause pump mechanism 224 to deliver doses of the drug or therapeutic agent to a user at predetermined intervals or as needed to bring blood glucose measurement values to a target blood glucose value based on the insulin infusion process according to some embodiments.

The devices in system 250, such as management device 206, wearable drug delivery device 202, and sensor 204, may also be operable to perform various functions including controlling wearable drug delivery device 202. For example, management device 206 may include a communication interface device 264, a processor 261, and a management device memory 263. In some embodiments, management device memory 263 may store an instance of insulin delivery application 219.

In some embodiments, sensor 204 of system 250 may be a continuous glucose monitor (CGM) or a manual glucose sensor, that may include a processor 241, a memory 243, a sensing or measuring device 244, and/or a communication interface device 246. Memory 243 may store an instance insulin delivery application 219 as well as other programming code and may be operable to store data related to insulin delivery application 219.

Instructions for determining the delivery of the drug or therapeutic agent (e.g., as a bolus dosage) to the user (e.g., the size and/or timing of any doses of the drug or therapeutic agent) may originate locally by wearable drug delivery device 202 or may originate remotely and be provided to wearable drug delivery device 202. In an example of a local determination of drug or therapeutic agent delivery, programming instructions, such as an instance of the insulin delivery application 219, stored in the memory 223 that is coupled to wearable drug delivery device 202 may be used to make determinations by wearable drug delivery device 202. In addition, wearable drug delivery device 202 may be operable to communicate via communication interface device 226 and wireless communication link 288 with wearable drug delivery device 202 and with blood glucose sensor 204 via communication interface device 226 and wireless communication link 287.

In addition or alternatively, remote instructions may be provided to wearable drug delivery device 202 over a wired or wireless link by the management device (PDM) 206. For example, PDM 206 may be equipped with a processor 261 that may execute an instance of the insulin delivery application 219 resident in the memory 263. Wearable drug delivery device 202 may execute any received instructions (originating internally or from management device 206) for the delivery of insulin to the user. In this manner, the delivery of the insulin to a user may be automated.

Devices within insulin delivery system 250 may be configured to communicate via various wired links 277-279 and/or wireless links 286-289. Wired links 277-279 may be any type of wired link provided by any known or future wired communication standard. Wireless links 286-289 may be any type of wireless link provided by any known or future wireless standard. As an example, wireless links 286-289 may enable communications between wearable drug delivery device 202, management device 206, sensor 204 based, and/or mobile device 216 on, for example, Bluetooth®, Wi-Fi®, a near-field communication standard, a cellular standard, or any other wireless optical or radio-frequency protocol. In some embodiments, mobile device 216 may operate as a management device 206 (for instance, management device 206 may not be a separate PDM device; rather, PDM functions are performed via insulin delivery application 219 operating on mobile device 216).

Although sensor 204 is depicted as separate from wearable drug delivery device 202, in various examples, sensor 204 and wearable drug delivery device 202 may be incorporated into the same unit. For example, sensor 204 may be a part of wearable drug delivery device 202 and contained within the same housing of wearable drug delivery device 202. Blood glucose measurement information (whether automatically or manually (fingerstick) determined) determined by sensor 204 may be provided to wearable drug delivery device 202 and/or management device 206, which may use the measured blood glucose values to determine an infusion amount or rate based on an insulin infusion process according to some embodiments.

In some examples, wearable drug delivery device 202 and/or management device 206 may include a user interface 227 and 268, respectively, such as a keypad, a touchscreen display, levers, buttons, a microphone, a speaker, a display, or the like, that is operable to allow for user input and/or output to user (for instance, a display of information). The wearable drug delivery device 202 may further include a heart rate sensor 257.

In some embodiments, drug delivery system 250 may implement an AP or AID algorithm (for instance, insulin delivery application 219) to govern or control automated delivery of insulin to a user based on an insulin infusion process according to some embodiments. Insulin delivery application 219 may be used to determine the times and dosages of insulin delivery (for example, a rate based on the basal parameter, I_{add}, adjustment factors, safety constraints, and/or the like). In various examples, the insulin delivery application 219 may determine the times and dosages for delivery based, at least in part, on information known about the user, such as gender, age, weight, height, and/or other information gathered about a physical attribute or condition of the user (e.g., from the sensor 204).

Included herein are one or more logic flows representative of exemplary methodologies for performing novel aspects of the disclosed architecture. While, for purposes of simplicity of explanation, the one or more methodologies shown herein are shown and described as a series of acts, those skilled in the art will understand and appreciate that the methodologies are not limited by the order of acts. Some acts may, in accordance therewith, occur in a different order and/or concurrently with other acts from that shown and described herein. For example, those skilled in the art will understand and appreciate that a methodology could alternatively be represented as a series of interrelated states or events, such as in a state diagram. Moreover, not all acts illustrated in a methodology may be required for a novel implementation.

A logic flow may be implemented in software, firmware, hardware, or any combination thereof. In software and firmware embodiments, a logic flow may be implemented by computer executable instructions stored on a non-transitory computer readable medium or machine readable medium. The embodiments are not limited in this context.

FIG. 3 illustrates an embodiment of a logic flow 300. Logic flow 300 may be representative of some or all of the operations executed by one or more embodiments described herein, such as devices of operating environments 100 and/or 200. In some embodiments, logic flow 300 may be representative of some or all of the operations of an insulin infusion process according to some embodiments.

At block 302, logic flow 300 may determine patient information. For example, patient 150 or caregiver for patient 150 may enter patient information 134 into computing device 110, for instance, via a user interface of insulin delivery application 140. In another example, insulin delivery application 140 may access patient information stored in data sources 192a-n, such as electronic medical records. In various embodiments, patient information 134 may include patient demographic information, physiological information, and/or insulin dosage information. For example, patient 150 may be a T2D patient using MDI-based therapy to manage their diabetic condition. In such an example, patient information 134 may include MDI information for patient 150.

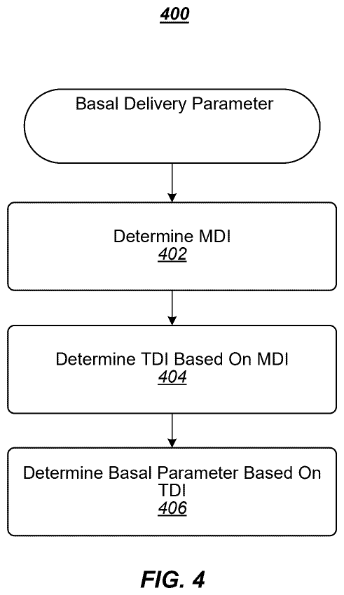

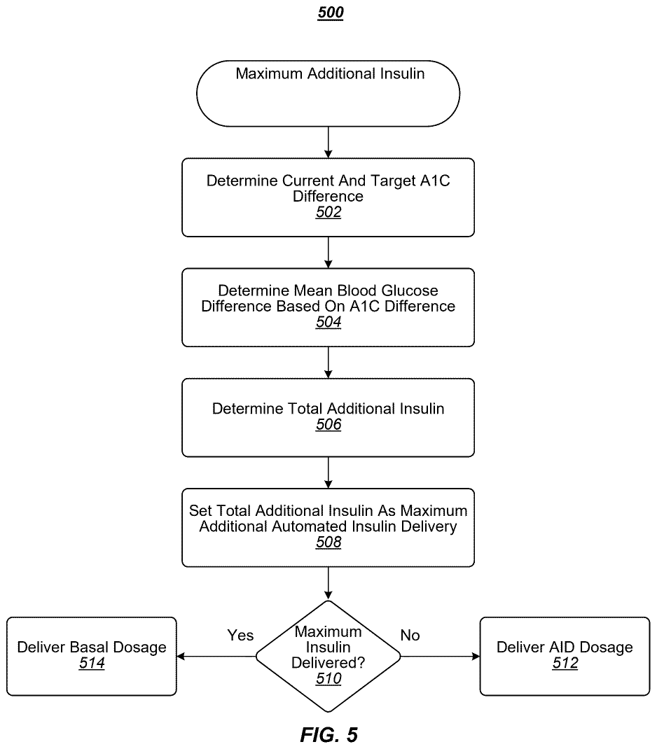

Logic flow 300 may determine insulin dosage information at block 304. For example, insulin delivery application 140 may determine TDI for patient 150 based on MDI information, for instance, via Equation (1), and then determine a basal parameter for patient 150 based on the TDI, for instance, according to Equation (2) (see, for example, FIG. 4). In addition, insulin delivery application 140 may determine the maximum daily additional insulin (I_{add}) for patient, for example, based on current and target A1C levels for patient 150 via Equation (5) (see, for example, FIG. 5). In various embodiments, insulin delivery application 140 may use the basal parameter and I_{add} to determine insulin dosage information 136, such as an infusion rate, an injection volume for an injection cycle, and/or the like.

At block 306, logic flow 300 may determine dosage adjustments. For example, insulin delivery application 140 may determine safety constraints for the insulin infusion process, such as an AID algorithm for determining that automated insulin dosage for an infusion cycle. Non-limiting examples of safety constraints may include a duration maximum safety constraint (see, for instance, Equation (8)) and/or an IOB safety constraint (see, for example, Equation (9)).

Logic flow 300 may administer the insulin dosage at block 308. For example, insulin delivery application 140 may instruct, manage, control, or otherwise cause AID device 160 to deliver a dosage of insulin to patient 150 determined based on the insulin infusion process according to some embodiments. The insulin dosage may include a volume of insulin (which may include no insulin or a volume of 0 units) determined based on the basal parameter, I_{add}, adjustment factors, safety constraints, and/or processes of an AID algorithm.

FIG. 4 illustrates an embodiment of a logic flow 400. Logic flow 400 may be representative of some or all of the operations executed by one or more embodiments described herein, such as devices of operating environments 100 and/or 200. In some embodiments, logic flow 400 may be representative of some or all of the operations of an insulin infusion process for determining a basal parameter according to some embodiments.

At block 402, logic flow 400 may determine MDI. For example, insulin delivery application 140 may receive patient information indicating MDI for patient 150, for instance, according to a T2D treatment regimen for patient 150. The MDI information may be entered manually by patient 150 or a caregiver of patient 150 and/or may be determined by insulin delivery application 140 based on patient characteristics, physician instructions or recommendations, treatment protocols, and/or the like. For example, patient 150 may have an MDI treatment plan of two injections of 20 units of long-acting insulin and 1 injection of 10 units of long-acting insulin.

Logic flow 400 may determine TDI based on MDI at block 404. For example, insulin delivery application 140 may use Equation (1) to determine TDI based on MDI for patient 150. For example, for an MDI of two injections of 20 units of long-acting insulin and 1 injection of 10 units of long-acting insulin, Equation (1) may determine a TDI of 50 units of insulin. At block 406, logic flow 400 may determine a basal parameter based on TDI. For example, insulin delivery application 140 may use Equation (2) to determine a basal parameter based on the TDI determined at block 404. In an example, for a TDI of 50 units, the basal parameter may be 50/24=2.08 units/hour. In some embodiments, the basal parameter may be provided to an AID algorithm or process to determine a volume of insulin to inject into patient 150 via AID device 160 (such as 2.08 units every hour cycle, 1.04 units every half-hour cycle, and so on).

FIG. 5 illustrates an embodiment of a logic flow 500. Logic flow 500 may be representative of some or all of the operations executed by one or more embodiments described herein, such as devices of operating environments 100 and/or 200. In some embodiments, logic flow 500 may be representative of some or all of the operations of an insulin infusion process for determining a maximum additional insulin value according to some embodiments.

At block 502, logic flow 500 may determine a difference between a current A1C value and a target A1C value. For example, patient 150 may have a current A1C value of 9% (for instance, measured via a blood test and entered manually via computing device and/or accessed via electronic medical records of data sources 192a-n) and a target A1C of 7% for a current A1C−target A1C difference of 2 (or 2%). Logic flow 500 may determine a mean blood glucose difference based on the A1C difference at block 504. For example, insulin delivery application 140 may use Equation (3) to determine the mean blood glucose difference (BG_{diff}). Logic flow 500 may determine the additional insulin at block 506. For example, insulin delivery application 140 may use Equation (5) to determine I_{add} based on BG_{diff} and CF (determined using Equation (4)).

At block 508, logic flow 500 may set the total additional insulin as the maximum additional automated insulin delivery. For example, I_{ADD} may be set as the maximum additional delivery of insulin allowed beyond the basal parameter that may be delivered by AID device 160 (for example, for bolus needs). Logic flow 500 may determine whether a maximum amount of insulin has been delivered at block 510. For example, at a delivery time or cycle t, insulin delivery application 140 may use Equation (6) to determine whether a maximum amount of insulin would be injected into patient 150 at time t. For example, in reference to Equation (6), if

$${\sum }_{i = 1}^{t} {AID}_{alg} ( t ) - basal ( t ) \leq basal * t$$

evaluates to true, then the maximum amount of insulin has not been injected into patient 150 and the AID_{alg}(t) value may be used at block 512. Alternatively, in reference to Equation (6), if

$${\sum }_{i = 1}^{t} {AID}_{alg} ( t ) - basal ( t ) > basal * t$$

evaluates to true, then the maximum amount of insulin has been injected into patient 150 and the basal amount of insulin b(t) is used as I_{AID}(t) at block 514.

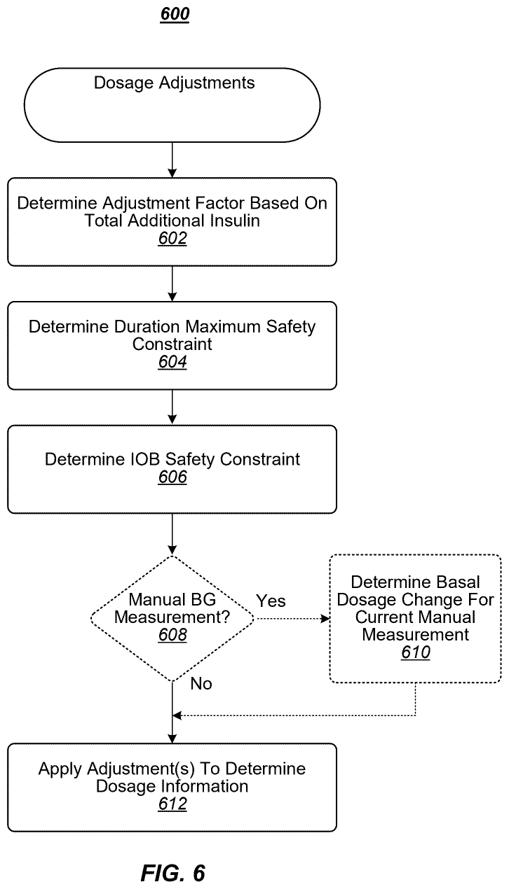

FIG. 6 illustrates an embodiment of a logic flow 600. Logic flow 600 may be representative of some or all of the operations executed by one or more embodiments described herein, such as devices of operating environments 100 and/or 200. In some embodiments, logic flow 600 may be representative of some or all of the operations of an insulin infusion process for determining dosage adjustments according to some embodiments.

At block 602, logic flow 600 may determine an adjustment factor based on total additional insulin. For example, insulin delivery application 140 may determine F_{S} based on Equation (7). In some embodiments, F_{S} may be used to adjust (for example, relax) safety constraints used to determine an insulin delivery volume at a particular delivery cycle, for instance, via an AID algorithm.

Logic flow 600 may determine a duration maximum threshold at block 604. For example, insulin delivery application 140 may determine a duration maximum threshold (or integral delivery) safety constraint via Equation (8). In some embodiments, the duration maximum threshold may specify that the total insulin delivery for patient 150 during a certain duration (for example, 3 hours) cannot exceed a tunable factor (for instance, 9) times the basal parameter. At block 606, logic flow 600 may determine an IOB threshold. For example, insulin delivery application 140 may determine an IOB based on Equation (9). In exemplary embodiments, the IOB safety constraint may include determining insulin delivery in deviations from basal as a baseline.

At block 612, logic flow 600 may apply the dosage adjustments to determine dosage information. For example, insulin delivery application 140 may apply the safety constraints determined in blocks 604 and 606 to determine a dosage amount for an infusion cycle based, for instance, on application of an AID algorithm. The dosage amount may be administered to patient 150.

In some embodiments, logic flow 600 may be used to manage manual blood glucose (BG) measurements. For example, at block 608, logic flow 600 may determine whether manual BG measurements are being used by AID device 160 (for instance, fingerstick-based measurements). If manual BG measurements are being used, logic flow 600 may determine a basal dosage change for a current manual measurement at block 610. For example, insulin delivery application 140 may determine Δb(i), which represents a change in basal of patient 150 based on a current manual or fingerstick BG measurement, via Equation (10). In some embodiments, Δb(i) may be made asymmetric and/or dependent on the current blood glucose of patient 150, for example, based on Equation (12). In various embodiments, an adjustment based on Δb(i) may be used by logic flow 600 to apply adjustments to determine dosage information at block 612.

While the present disclosure has been illustrated and described in detail in the drawings and foregoing description, the same is to be considered as illustrative and not restrictive in character, it being understood that only the certain embodiments have been shown and described and that all changes, alternatives, modifications and equivalents that come within the spirit of the disclosure are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. An apparatus, comprising:
at least one memory; and
logic coupled to the at least one memory, the logic to:
determine a basal parameter for a patient based on a type 2 diabetes (T2D) multiple daily injection (MDI) information of the patient, the basal parameter indicating a basal infusion rate,
determine an additional insulin (I_{add}) value based on a mean blood glucose difference (BG_{diff}) information associated with the patient,
determine an insulin volume to infuse into the patient based on the basal parameter and I_{add}, and
administer the insulin volume to the patient.

2. The apparatus of claim 1, the logic to determine the basal parameter according to:
$$TDI = {\sum }_{n = 1}^{{n}_{{max}_{=}}} {MDI}_{n} , and$$
$$basel parameter = \frac{TDI}{t} ,$$
where t is an infusion time period.

3. The apparatus of claim 1, the logic to determine BG_{diff} based on a difference between a current A1C and a target A1C for the patient.

4. The apparatus of claim 3, the logic to determine the I_{add} value according to:
$${I}_{add} = \frac{B {G}_{diff}}{C F} * x ,$$
where x is a tunable cycle parameter configured to represent the number of cycles of insulin peak times per infusion time period,
where CF is determined according to:
$$CF = \frac{y}{z * basal} ,$$
where y is an insulin sensitivity factor and z is an infusion time period.

5. The apparatus of claim 1, the logic to adjust the insulin volume based on at least one safety constraint.

6. The apparatus of claim 6, the logic to determine the at least one safety constraint based on an adjustment factor (F_{S}) determined based on TDI and I_{add} according to:
$${F}_{s} = 1 + max ( 0 , \frac{{I}_{a d d}}{TDI} ) .$$

7. The apparatus of claim 5, the at least one safety constrain comprising a duration maximum safety constraint indicating a maximum volume of insulin that may be infused into the patient during a duration.

8. The apparatus of claim 5, the at least one safety constrain comprising an insulin-on-board (IOB) safety constraint indicating deviations from basal infusion for the patient.

9. The apparatus of claim 1, the logic to determine an insulin volume using an automatic insulin delivery (AID) process based on continuous glucose measurement (CGM) information of the patient.

10. The apparatus of claim 1, the logic to determine an insulin volume using an automatic insulin delivery (AID) process using manual blood glucose measurement information of the patient.

11. The apparatus of claim 10, the logic to determine an adjustment (Δb(i)) in insulin delivery responsive to receiving the manual glucose measurement information for the patient, wherein Δb(i) represents a change in basal insulin of the patient based on the manual glucose measurement information for the patient.

12. A method, comprising:
operatively coupling an automatic insulin delivery (AID) device to a patient;
performing, via a processor of a computing device operatively coupled to the AID device, an insulin infusion device by:
determining a basal parameter for the patient based on a type 2 diabetes (T2D) multiple daily injection (MDI) information of the patient, the basal parameter indicating a basal infusion rate,
determining an additional insulin (I_{add}) value based on a mean blood glucose difference (BG_{diff}) information associated with the patient, and
determining an insulin volume to infuse into the patient based on the basal parameter and I_{add}; and
administering the insulin volume to the patient via the AID device.

13. The method of claim 12, comprising determining BG_{diff} based on a difference between a current A1C and a target A1C for the patient.

14. The method of claim 13, comprising determining the I_{add} value according to:
$${I}_{add} = \frac{B {G}_{diff}}{C F} * x ,$$
where x is a tunable cycle parameter configured to represent the number of cycles of insulin peak times per infusion time period,
where CF is determined according to:
$$C F = \frac{y}{z * basal} ,$$
where y is an insulin sensitivity factor and z is an infusion time period.

15. The method of claim 12, comprising adjusting the insulin volume based on at least one safety constraint.

16. The method of claim 15, the at least one safety constrain comprising a duration maximum safety constraint indicating a maximum volume of insulin that may be infused into the patient during a duration.

17. The method of claim 15, the at least one safety constrain comprising an insulin-on-board (IOB) safety constraint indicating deviations from basal infusion for the patient.

18. The method of claim 12, comprising determining an insulin volume using an automatic insulin delivery (AID) process based on continuous glucose measurement (CGM) information of the patient.

19. The method of claim 12, comprising determining an insulin volume using an automatic insulin delivery (AID) process using manual blood glucose measurement information of the patient.

20. The method of claim 19, comprising determining an adjustment (Δb(i)) in insulin delivery responsive to receiving the manual glucose measurement information for the patient, wherein Δb(i) represents a change in basal insulin of the patient based on the manual glucose measurement information for the patient.
